(19) United States
(12) Patent Application Publication
(10) Pub. No.: US 2003/0191465 A1
(43) Pub. Date: Oct. 9, 2003

(54) Electrosurgicalscissors for endoscopic mucosal resection

(75) Inventors: Naohisa Yahagi, Tokyo (JP); Satoshi Kidooka, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation

(21) Appl. No.: 10/404,558

(22) Filed: Apr. 2, 2003

(30) Foreign Application Priority Data
Apr. 9, 2002 (JP) 2002-106008

(51) Int. Cl. A61B 18/14

(52) U.S. Cl. 606/48; 606/50

(57) ABSTRACT

An electrosurgicalscissors for an endoscopic mucosal resection includes an flexible inserting portion that is to be inserted into a body cavity through an endoscope and first and second electrodes mounted to the distal end of the inserting portion so as to be movable between an open position and a closed position. The first and second electrodes are connected with conductive wires to be provided with high frequency voltage. Each of said first and second electrodes as a contact surface formed in a substantially straight shape. The first and second electrodes come into contact with each other at the contact surfaces when moved to the closed position and the contact surfaces are spaced apart from each other when the first and second electrodes are moved to the open position.

### BACKGROUND OF THE INVENTION

The present invention relates to an electrosurgicalscissors for an endoscopic mucosal resection that utilizes high frequency electric current.

The endoscopic mucosal resection is often performed instead of an abdominal operation for removing a lesion within a human cavity such as an early gastric cancer.

If the lesion to be removed is relatively small or is forming a polyp, a snare, which is a looped wire electrode, is inserted into the human cavity through an endoscope for carrying out the endoscopic mucosal resection

However, if the lesion to be removed is larger than about 3 cm, for example, the mucosa is cut off by monopolar electrocautery which utilizes a needle or rod like shape electrode in combination with a large counter electrode placed on the body surface of the patient. High frequency electric current is generated between the needle or rod like shape electrode and the counter electrode to cauterize the tissue in the vicinity of the needle or rod like shape electrode.

The disadvantage of the monopolar electrocautery is that it may make a hole to the tissue that is much deeper than required if the high frequency current is generated continuously for a long time since the current flows from one electrode located inside the human body to the other electrode placed on the outer body surface.

Forming such a deep hole can be avoided by generating the current intermittently, however, this requires longer surgery.

Thus, there is a need for an electrocautery for endoscopic mucosal resection that removes the mucosa by means of high frequency current in a relatively short surgery without the danger of cutting deep into the mucosa.

### SUMMARY OF THE INVENTION

The present invention is advantageous in that an electrosurgicalscissors that satisfies the above mentioned need is provided.

According to an aspect of the invention, there is provided an electrosurgicalscissors for an endoscopic mucosal resection. The electrosurgicalscissors includes an flexible inserting portion that is to be inserted into a body cavity through an endoscope and first and second elongated electrodes mounted to the distal end of the inserting portion so as to be movable between an open position and a closed position.

The first and second electrodes are connected with conductive wires to be provided with high frequency voltage. Each of said first and second electrodes has a contact surface formed in a substantially straight shape. The first and second electrodes come into contact with each other at the contact surfaces when moved to the closed position and the contact surfaces are spaced apart from each other when the first and second electrodes are moved to the open position.

The electrosurgicalscissors arranged as above can pinch a portion of tissue such as mucosa between the first and second electrodes. If high frequency voltage is supplied to the first and second electrodes, high frequency current flows only through the tissue that is pinched between the first and second electrodes. Accordingly, the electrosurgicalscissors does not cut the tissue deeply even if the high frequency electric power is continuously supplied for a long time.

Optionally, the contact surfaces are formed flat, and the pair of electrodes make contact with each other at substantially the whole areas of the contact surfaces.

Alternatively, a plurality of protrusions are provided on the contact surfaces. The protrusion allows the first and second electrodes to firmly grasp the tissue. Optionally, the plurality of protrusions are arranged along a longitudinal direction of the contact surface. Further optionally, the plurality of protrusions are inclined toward a proximal end of said contact surface to prevent the tissue from slipping off the electrodes when the electrosurgicalscissors is retracted to remove the tissue.

Optionally, each of the first and second electrodes has a round distal end so that the electrosurgicalscissors can be smoothly introduced into a body cavity through an endoscope.

In some embodiments, the first and second electrodes are arranged to move between the open and closed positions along a common plane, and the contact surfaces are formed perpendicularly to the common plane, so that the first and second electrodes open and close like pincers or pliers.

In other embodiments, the first and second electrodes are arranged such that they open and close like scissors. That is, the first and second electrodes are arranged to move between the open and closed positions along first and second planes, respectively, which planes are parallel to and spaced apart from each other, and the contact surfaces are formed in parallel to the first and second planes.

The contact surfaces may be formed such that the first and second electrodes come into contact first at the distal ends thereof so that the electrosurgicalscissors can cut small area of the tissue with the distal ends of the first and second electrodes.

Alternatively, the contact surfaces may be formed such that the first and second electrodes come into contact first at the proximal ends thereof so that the electrosurgicalscissors can also mechanically cut a hard portion of the tissue.

### BRIEF DESCRIPTION OF THE ACCOMPANYING DRAWINGS

FIG. 1 schematically shows an electrosurgicalscissors according to a first embodiment of the invention connected to a high frequency power supply;

FIGS. 2A and 2B show partially sectional side views of the distal end portion of the electrosurgicalscissors shown in FIG. 1;

FIG. 2C is a sectional view of the electrosurgicalscissors shown in FIG. 2A observed from the direction indicated by the arrow A in FIG. 2A;

FIG. 2D is a sectional view of the electrosurgicalscissors shown in FIG. 2A taken along the line B-B;

FIG. 3 is a front view of the electrodes of the electrosurgicalscissors shown in FIG. 2A located at a closed position thereof;

FIG. 4 is a perspective view of the electrode of the electrosurgicalscissors shown in FIG. 2A;

FIGS. 5A and 5B respectively show a perspective view and a side view of a variation of the electrode shown in FIG. 4;

FIGS. 6A and 6B show partially sectional side views of a distal end portion of an electrosurgicalscissors according to a second embodiment of the invention;

FIG. 6C is a sectional view of the electrosurgicalscissors shown in FIG. 6A observed from the direction indicated by the arrow C in FIG. 6A;

FIG. 7 is a front view of the electrodes of the electrosurgicalscissors shown in FIG. 6A located at the closed position thereof;

FIGS. 8A and 8B show partially sectional side views of the distal end portion of an electrosurgicalscissors according to a third embodiment of the invention;

FIG. 8C is a sectional view of the electrosurgicalscissors shown in FIG. 8A observed from the direction indicated by the arrow D in FIG. 8A; and

FIG. 9 is a front view of the electrodes of the electrosurgicalscissors shown in FIG. 8A located at the closed position thereof.

### DETAILED DESCRIPTION OF THE EMBODIMENTS

Hereinafter, an embodiment of the invention will be described with reference to the accompanying drawings.

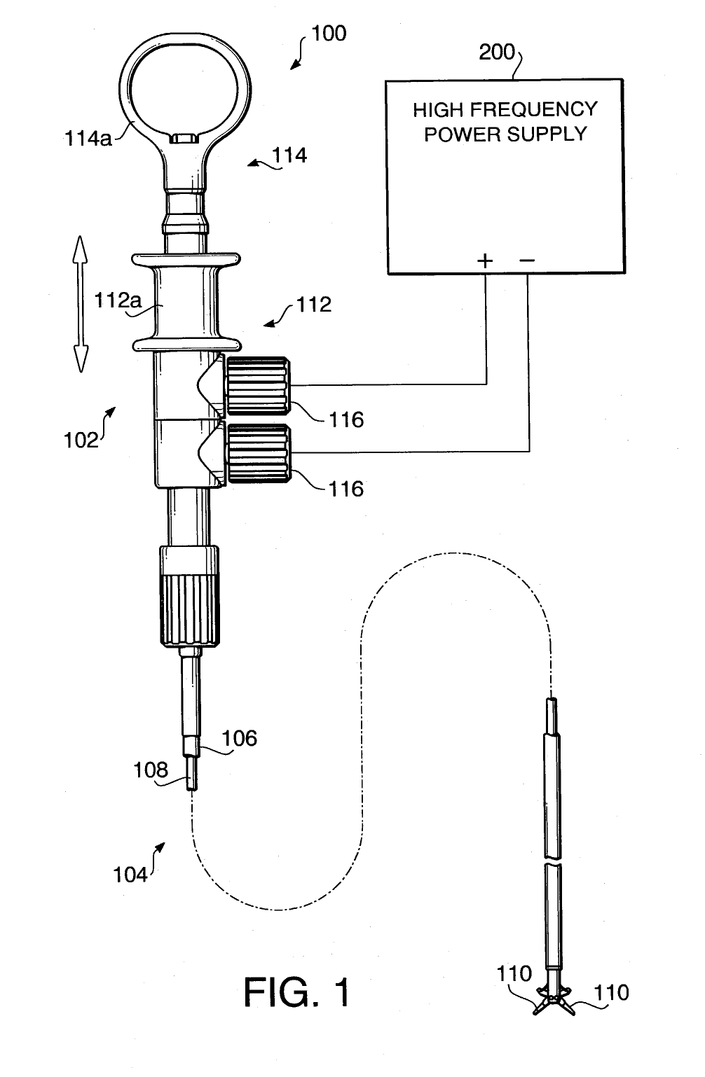

FIG. 1 schematically shows an electrosurgicalscissors 100 according to a first embodiment of the invention connected to a high frequency power supply 200.

The electrosurgicalscissors 100 includes an operation portion 102 and an inserting portion 104 connected to the distal end of the operation portion 102.

The inserting portion 104 is provided in a form and size that allows it to be introduced into a body cavity through a treatment tool inserting channel of an endoscope (not shown). The inserting portion 104 includes an elongated and flexible sheath 106, a pair of conductive wires 108 (only one is shown) slidably inserted through the sheath 106, and a pair of electrodes 110 provided at the distal end of the insertion portion 104 and connected to the conductive wires 108. The sheath 106 is made of insulating material such as poly-tetra-fluoro-ethylene (PTFE). In an exemplary embodiment, the sheath 106 is 1 m to 2 m long and has an outer diameter of 2 mm to 3 mm.

The operating portion 102 includes a cylindrical portion 112 and a rod portion 114 slidably inserted into the cylindrical portion 112.

The cylindrical portion 112 has a circumferential groove 112a at the proximal end thereof. A user of the electrosurgicalscissors 100 can hold the cylindrical portion 112 by pinching it at the groove 112a with his index finger and long finger.

The rod portion 114 has a ring 114a into which the user can insert his thumb to slide the rod portion 114 within the cylindrical portion 112 back and forth.

The rod portion 114 is connected with the pair of wires 108 in the cylindrical portion 112 such that the wires 108 retract and proceed in the sheath 106 as the rod portion 114 is moved back and forth with respect to the cylindrical portion 112. It should be noted that the pair of wires 108 may be fixed to each other so that they slide integrally within the sheath 106 to move the pair of electrodes 110 simultaneously.

The conductive wires 108 are detachable connected to the high frequency power supply 200 via a pair of connectors 116 provided to the side surface of the cylindrical portion 112. One of the conductive wires 108 is connected to the positive terminal of the power supply 200 and the other to the negative terminal.

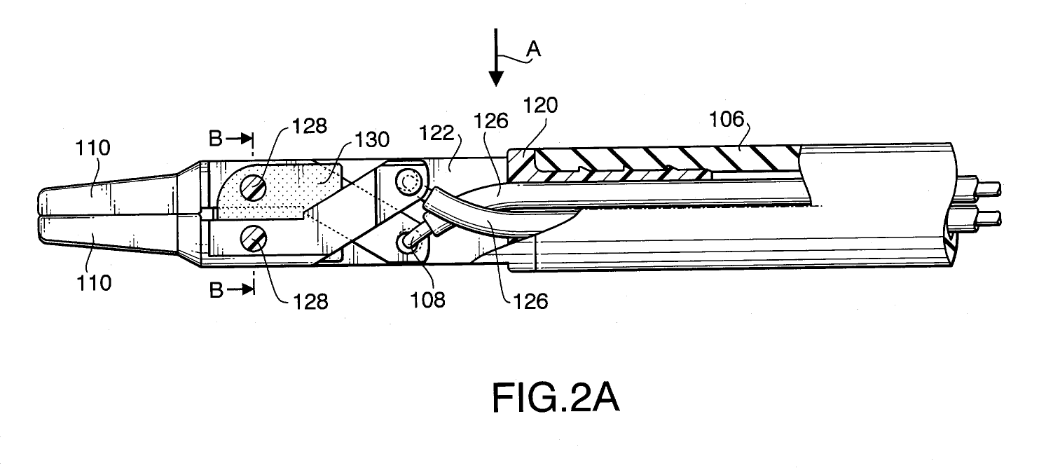

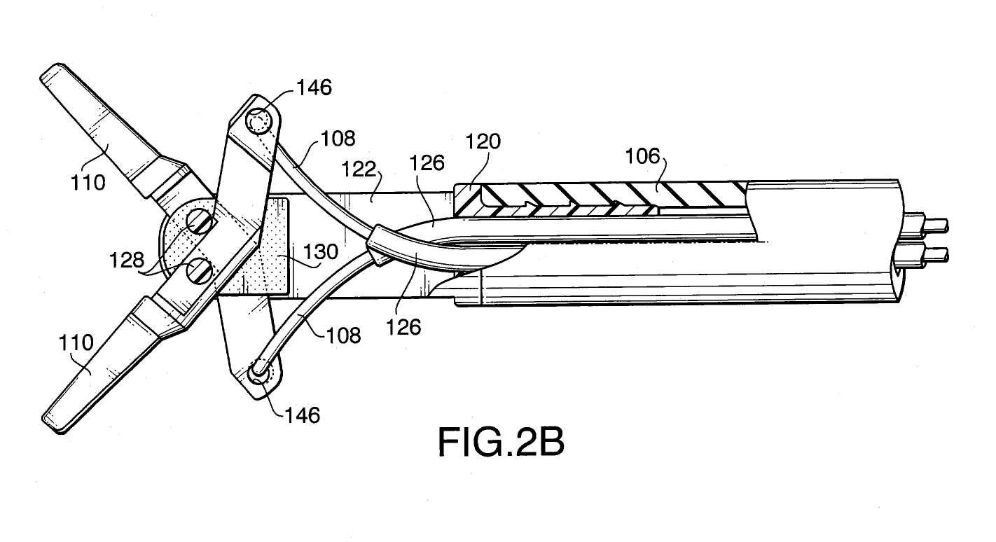

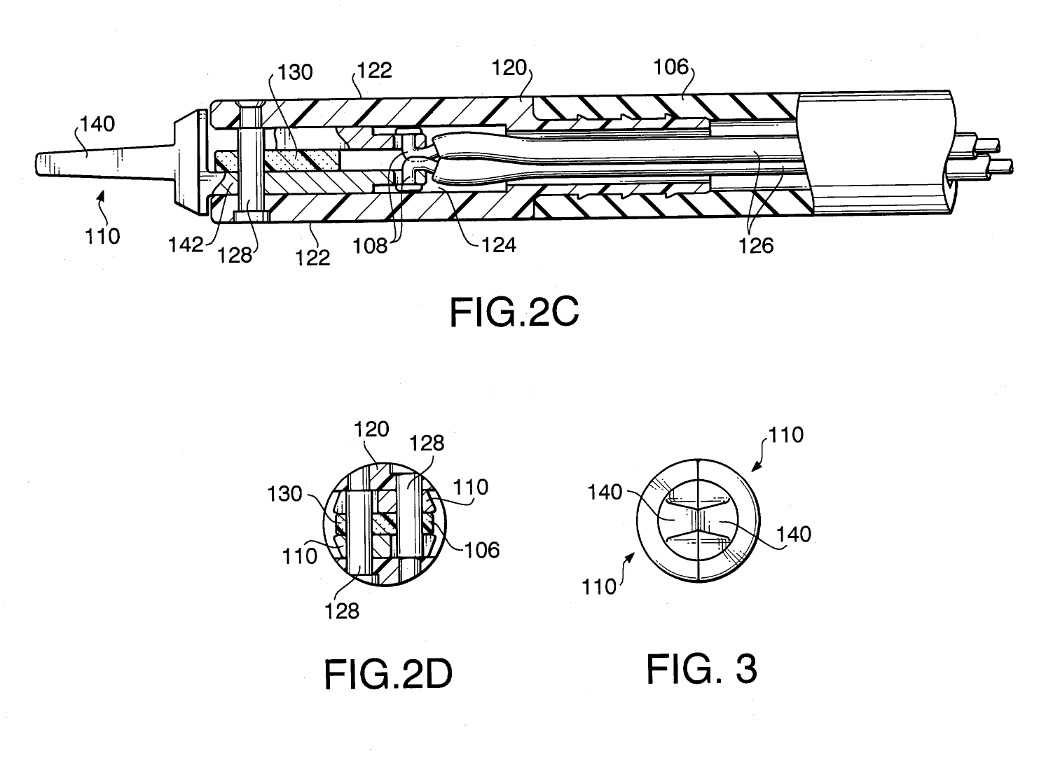

FIGS. 2A and 2B show partially sectional side views of the distal end portion of the electrosurgicalscissors 100 shown in FIG. 1. Note that the pair of electrodes 110 are located at a closed position in FIG. 2A and at an open position in FIG. 2B. FIG. 2C is a sectional view of the electrosurgicalscissors 100 shown in FIG. 2A observed from the direction indicated by the arrow A in FIG. 2A. Note that FIG. 2C is drawn as a composite view combining cross sectional views at various positions. FIG. 2D is a sectional view of the electrosurgicalscissors 100 shown in FIG. 2A taken along the line B-B, and FIG. 3 is a front view of the pair of electrodes 110 shown in FIG. 2A.

As shown in FIGS. 2A through 2C, a supporting member 120 for supporting the pair of electrodes 110 is mounted to the distal end of the flexible sheath 106. The supporting member 120 is made of hard insulating material such as rigid plastic. The supporting member 120 has two arms 122 extending forwards in parallel to each other to form a slit 124 having a constant width.

Two pins 128 are supported between the arms 122 in the vicinity of the distal end thereof. The pins 128 are arranged parallel to and spaced apart from each other, and perpendicular to the side walls of the slit 124. The pins 128 are made of stainless steel, for example.

The pair of electrodes 110 are partially inserted into the slit 124 of the supporting member 120 and rotatably mounted to the pair of pins 128. Thus, the pair of electrodes 110 can move between the closed position shown in FIG. 2A, at which the electrodes 110 come in contact with each other, and the open position shown in FIG. 2B. at which the electrodes 110 are located apart from each other.

The rear ends or proximal ends of the electrodes 110 are connected with the conductive wires 108. Each of the conductive wires 108 is covered with an insulating tube 126 except the end portion thereof at which the conductive wire 108 is connected to the corresponding electrode 110.

An insulating block 130 is located in the slit 124 of the supporting member 120 to prevent the electrodes 110 from coming into contact to each other within the slit 124. The insulating block 130 is located between the electrodes 110 and supported by the pins 128. The insulating block is made of resin such as poly-tetra-fluoro-ethylene, for example.

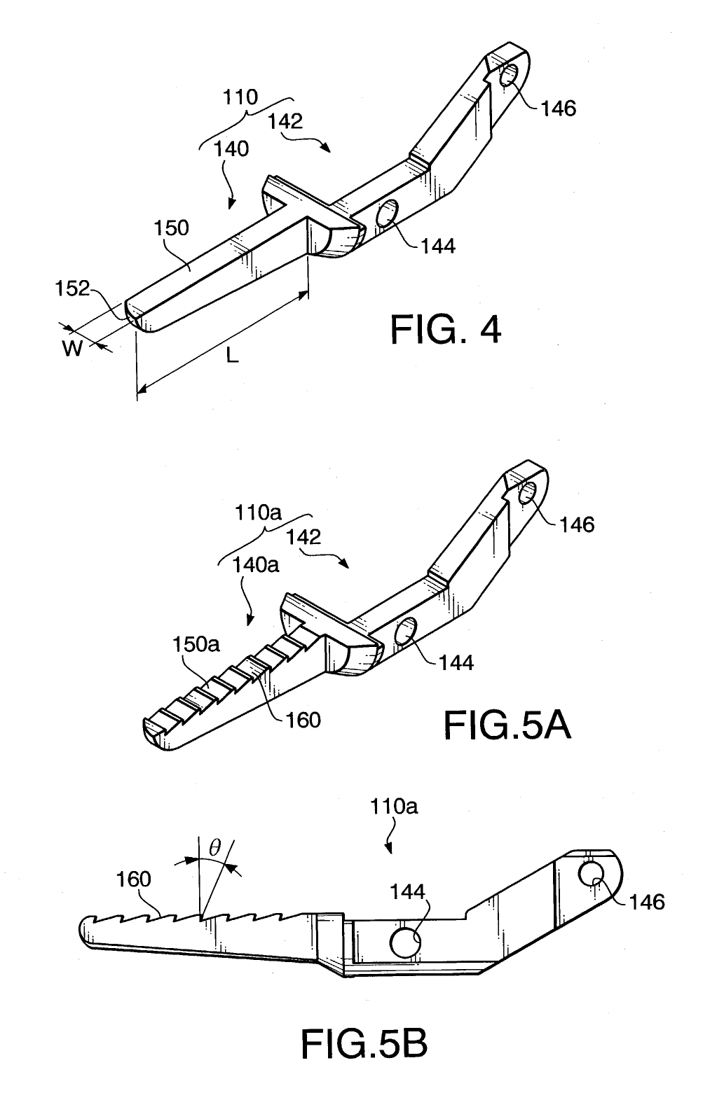

FIG. 4 is a perspective view of the electrode 110. The electrode 110 is an elongated member made of metal such as stainless steel. The electrode 110 includes an elongated front (distal) portion 140 and an elongated rear (proximal) portion 142. When the electrode 110 is mounted to the supporting member 120, the front portion 140 is located in front of the arms 122 and the back portion 142 between the arms 122 (see FIG. 2C).

Two through holes are formed to the back portion 142 of the electrode 110. The first one is a supporting hole 144 located at substantially the center of the electrode 110. The other one is a connection hole 146 formed in the vicinity of the rear end of the electrode 110.

As shown in FIG. 2D, the electrode 110 is pivotably mounted to the supporting member 120 by inserting the corresponding pin 128 through the supporting hole 144. Thus, the electrode can swing between the closed position shown in FIG. 2A and the opened position shown in FIG. 2B.

As can be seen in FIG. 2B, the distal end of the conductive wire 108, which is exposed from the insulating tube 126, is passed through the connecting hole 146 to be connected with the electrode 110.

The rear portion 142 of the electrode 110 is slightly bent so that the conductive wire 108 sliding back and forth within the sheath 106 can swing the electrode 110 around the pin 128 between the open and closed positions.

The front portion 140 of each electrode 110 is provided with a flat surface 150 that has a straight shape. The flat surface 150 is formed so as to be perpendicular to a plane along which the electrode 110 moves between the open and closed positions. The flat surface 150 is also formed such that it comes into contact with the contact surface of the other electrode 110 at substantially the whole area thereof.

The front portion 140 is formed such that the width gradually decreases toward the front end 152 thereof. Further, the front end 152 of the electrode 140 is rounded to allow smooth insertion of the electrosurgicalscissors 100 through the treatment tool insertion channel of an endoscope.

In an exemplary embodiment of the invention, the length L of the front portion 140 is within the range of 2 mm to 3 mm and width W at the front end is within the range 0.2 mm to 0.3 mm. The electrode 110 having such a dimension is easy to handle in the body cavity under an endoscopic observation, and facilitates the resection of mucosa.

As can be seen in FIG. 2C, the front portion 140 of each of the electrodes 110 is displaced from the rear portion 142 in a width direction thereof such that the front portions 140 of both of the electrodes 110 swing along a common plane and move between the open and closed positions like pincers or pliers (see also FIG. 3).

The insertion portion 104 of the electrosurgicalscissors 100 configured as above is introduced into a body cavity such as a stomach through an endoscope and the pair of electrodes 110 are located in the vicinity of the target portion of the mucosa to be removed.

Then, the operation portion 102 of the electrosurgicalscissors 100 is operated so as to push the rod portion 114 into the cylindrical portion 112. As a result, the pair of conductive wires 108 are slid forwards within the sheath 106 and swing the pair of electrodes 110 to the open position shown in FIG. 2B. Then, the pair of electrodes 110 are moved by the endoscope to locate the target portion of the mucosa between the electrodes 110.

Next, the pair of conductive wires 108 are retracted by pulling back the rod portion 114 with respect to the cylindrical portion 112 to move the electrodes 110 to the closed position and thereby grasp the target mucosa between the front portions 140 of the electrodes 110.

Next, a high frequency electrical power is supplied from the power supply 200 to the pair of electrodes 110 via the conductive wires 108. As a result, a high frequency current flows through the mucosa placed between the electrodes 110 to coagulate and/or cut the mucosa.

It should be noted that the electric current flows only through the tissue that is pinched between the pair of electrodes and hence the tissue under the mucosa placed between the electrodes will not be damaged. As a result, electrosurgicalscissors 100 according to the embodiment of the invention does not cut deep into the tissue even if the high frequency electric power is continuously supplied for a long time.

Further, since the tissue are well coagulated with the heat of the electric current, significant bleeding does not occur and the target tissue can be removed smoothly. Thus, the electrosurgicalscissors 100 according to the embodiment of the invention can realize a safety and quick endoscopic mucosal resection.

FIGS. 5A and 5B respectively show a perspective view and a side view of an electrode 110a which is a variation of the electrode 110 shown in FIG. 4. The electrode 110a shown in FIGS. 5A and 5B differs from the electrode 110 shown in FIG. 4 in that a plurality of protrusions 160 are formed on the contact surface 150a. Except the above, the electrode 110a shown in FIGS. 5A and 5B has substantially the same configuration as the electrode 110 shown in FIG. 4.

The plurality of protrusions 160 are substantially aligned along the longitudinal direction (or back and forth direction) of the electrode 110a. Further, each protrusion 160 is inclined toward the rear side of the electrode 110a, that is, the side surface at the rear side of the protrusion 160 is inclined toward the rear side of the electrode 110a for an angle θ which is, in an exemplary embodiment of the invention, within a range of 5 to 15 degree.

The plurality of protrusions 160 allows the pair of electrodes 110a to grasp the mucosa firmly. In particular, since the protrusions 160 are inclined toward the rear side of the electrode 110a, the protrusions 160 prevent the target mucosa from slipping out from between the pair of electrodes 110a when the electrosurgicalscissors 100 is retracted to pull and thereby separate the target mucosa from the submucosa.

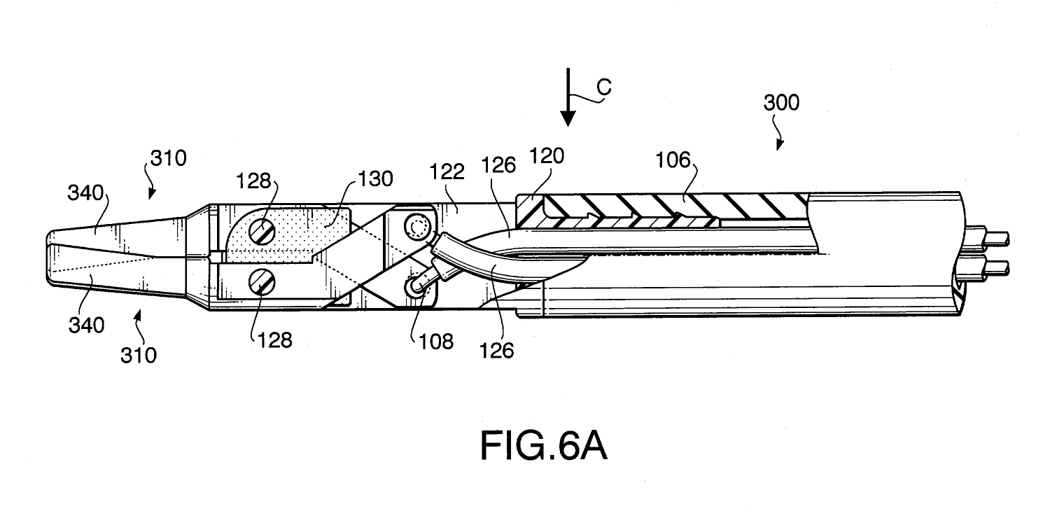

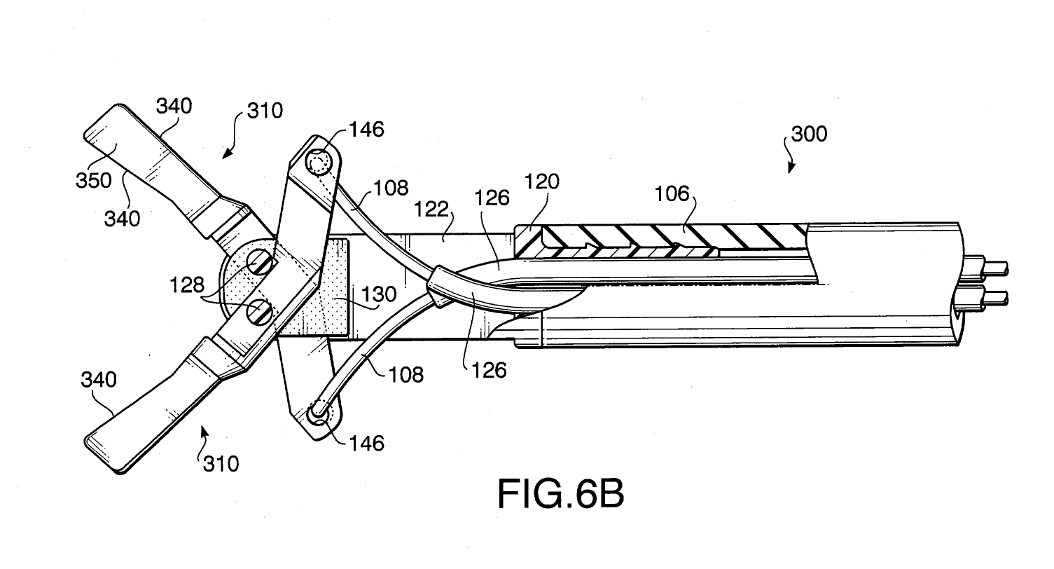

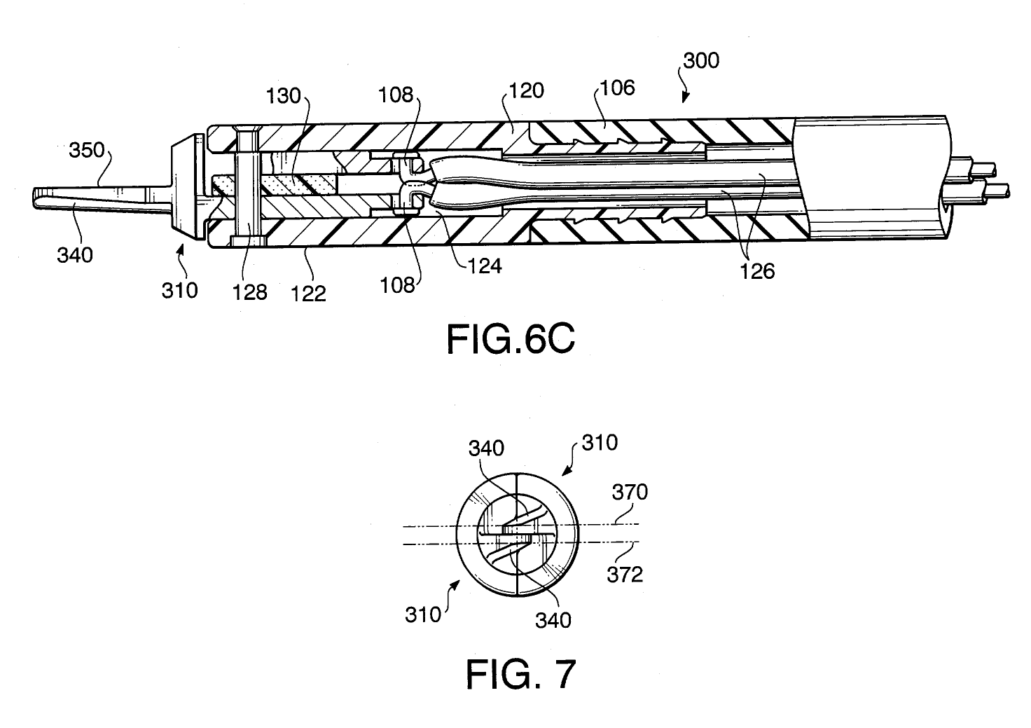

FIGS. 6A and 6B show partially sectional side views of a distal end portion of an electrosurgicalscissors 300 according to a second embodiment of the invention. The electrosurgicalscissors 300 shown in FIGS. 6A and 6B has a pair of electrodes 310 formed in different shapes than that of the pair of electrodes 110 of the electrosurgicalscissors 100 shown in FIGS. 2A and 2B. Except the above, the electrosurgicalscissors 300 shown in FIGS. 6A and 6B has substantially the same configuration as the electrosurgicalscissors 100 shown in FIGS. 2A and 2B. Note that the pair of electrodes 110 are located at the closed position in FIG. 6A and at the open position in FIG. 6B.

FIG. 6C is a sectional view of the electrosurgicalscissors 300 shown in FIG. 6A observed from the direction indicated by the arrow C in FIG. 6A. Note that FIG. 6C is drawn as a composite view combining cross sectional views at various positions. FIG. 7 is a front view of the pair of electrodes 310 located at the closed position thereof.

The pair of electrodes 310 of the electrosurgicalscissors 300 of the second embodiment has substantially the same configuration as that of the electrodes 110 of the electrosurgicalscissors 100 of the first embodiment except the form of the front portions 340 thereof. The electrodes 310 are formed such that the front portions 344 thereof move between the open and closed positions along different planes 370 and 372 (shown by chain double-dashed lines in FIG. 7) that are parallel to and spaced apart from each other. The front portion 340 of each electrode 310 is provided with a flat side surface 350 which is parallel to the planes 370 and 372 along which the electrodes 310 move. When the electrodes 310 are moved to the closed position, the front portions 340 thereof come into contact with each other at the flat side surfaces 350, as shown in FIG. 7, like a pair of scissors.

Each of the flat side surface 350 has a larger width at the front or distal end thereof than at the rear or proximal end thereof. Accordingly, the electrodes 340 comes into contact first at the distal ends thereof as the electrodes 340 are moved to the closed position.

The electrosurgicalscissors 300 having the pair of electrodes 310 configured as above allows the resection of small target portion of the mucosa by pinching the target portion at the tip end of the electrodes.

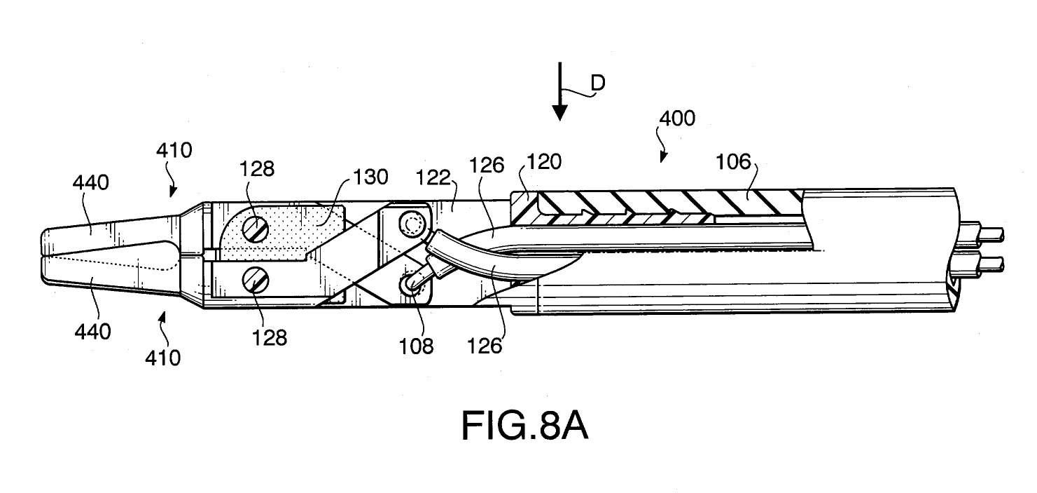

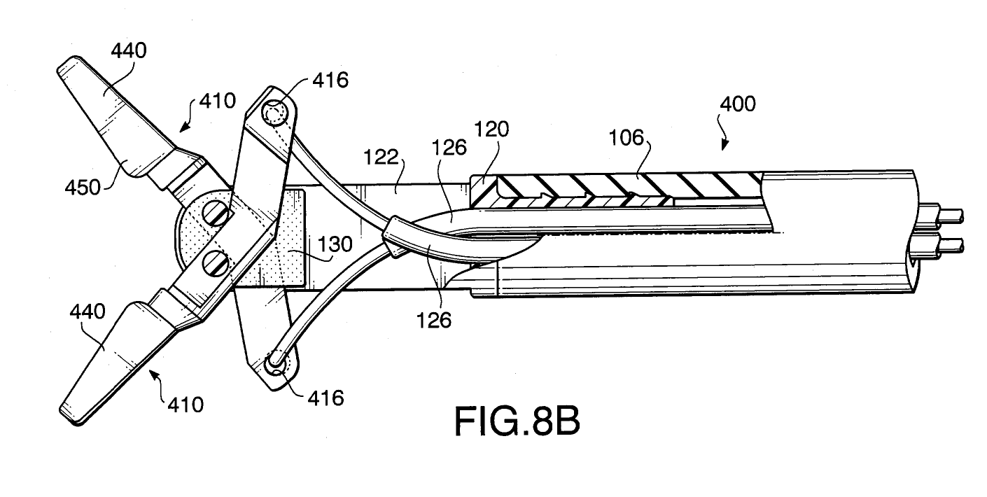

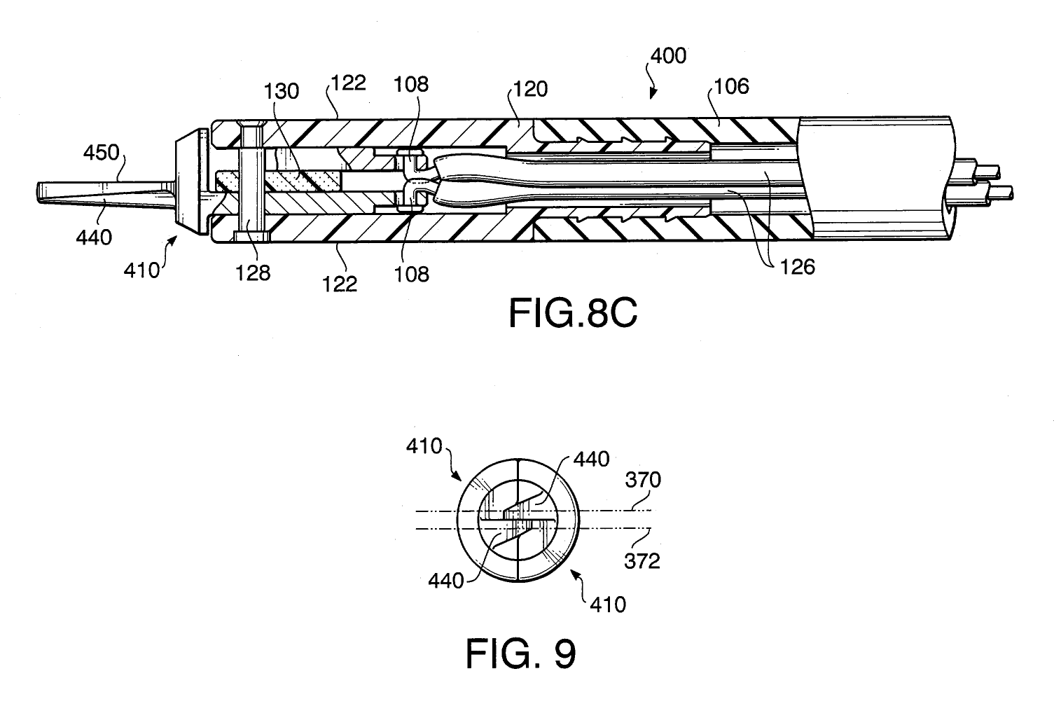

FIGS. 8A and 8B show partially sectional side views of a distal end portion of an electrosurgicalscissors 400 according to a third embodiment of the invention. The electrosurgicalscissors 400 shown in FIGS. 8A and 8B is provided with a pair of electrodes 410 which are a variation of the pair of electrodes 310 of the second embodiment of the invention. Except the above, the electrosurgicalscissors 400 according to the third embodiment has substantially the same configuration as the electrosurgicalscissors 300 of the second embodiment. Note that the pair of electrodes 410 are at the closed position in FIG. 8A and at the open position in FIG. 8B.

FIG. 8C is a sectional view of the electrosurgicalscissors 400 shown in FIG. 8A observed from the direction indicated by the arrow D in FIG. 8A. Note that FIG. 8C is drawn as a composite view combining cross sectional views at various positions. FIG. 9 is a front view of the distal end of the pair of electrodes 410 located at the closed position thereof.

The pair of electrodes 410 of the third embodiment has substantially the same configuration as the electrodes 310 of second embodiment except that the flat side surface 450 of each electrode 410 has a larger width at the rear or proximal end thereof than at the front or distal end thereof (see FIGS. 8A and 8B). Accordingly, the electrodes 440 comes into contact to each other first at the rear ends of the front portions 450 as the electrodes 440 are moved to the closed position.

The electrosurgicalscissors 400 provided with the pair of electrodes 410 configured as above has the advantage that is also can mechanically cut relatively hard tissue with the rear sides of the front portions 440 of the electrodes 410.

The present disclosure relates to the subject matter contained in Japanese Patent Application No. P2002-106008, filed on Apr. 9, 2002, which is expressly incorporated herein by reference in its entirety.

What is claimed is:

1. An electrosurgicalscissors for an endoscopic mucosal resection, comprising:
an flexible inserting portion that is to be inserted into a body cavity through an endoscope; and
first and second elongated electrodes mounted to the distal end of said inserting portion so as to be movable between an open position and a closed position, said first and second electrodes being connected with conductive wires to be provided with high frequency voltage, each of said first and second electrodes having a contact surface formed in a substantially straight shape, said first and second electrodes come into contact with each other at said contact surfaces when moved to said closed position, said contact surfaces being spaced apart from each other when said first and second electrodes are moved to said open position.

2. The electrosurgicalscissors according to claim 1, wherein said contact surfaces are formed flat, said pair of electrodes make contact with each other at substantially the whole areas of said contact surfaces.

3. The electrosurgicalscissors according to claim 1, wherein a plurality of protrusions are provided on said contact surfaces.

4. The electrosurgicalscissors according to claim 3, wherein said plurality of protrusions are arranged along a longitudinal direction of said contact surface.

5. The electrosurgicalscissors according to claim 3, wherein said plurality of protrusions are inclined toward a proximal end of said contact surface.

6. The electrosurgicalscissors according to claim 1, wherein each of said first and second electrodes has a round distal end.

7. The electrosurgicalscissors according to claim 1, wherein said first and second electrodes move between said open and closed positions along a common plane, and
wherein said contact surfaces are formed perpendicularly to said common plane.

8. The electrosurgicalscissors according to claim 1, wherein said first and second electrodes move between said open and closed positions along first and second planes, respectively, said first and second planes being parallel to and spaced apart from each other, and
wherein said contact surfaces are formed in parallel to said first and second planes and such that said first and second electrodes come into contact first at the distal ends thereof.

9. The electrosurgicalscissors according to claim 1, wherein said first and second electrodes move between said open and closed positions along first and second planes, respectively, said first and second planes being parallel to and spaced apart from each other, and
wherein said contact surfaces are formed in parallel to said first and second planes and such that said first and second electrodes come into contact first at the proximal ends thereof.
